SEQUENCE LISTING (18 sequences)

SEQ ID NO: 1 - DNA, 615 bp, Bacillus subtilis
atgaaagcagaattcaagcgtaaaggagggggcaaagtgaaactcgttgtcggaatgacaggggcaacaggggccattttcggggtcaggctgctgcagtggctgaaggccgccggagtggaaacccatctcgttgtgtctccttgggcaaacgtcacgatcaaacacgaaacaggctatacgttacaagaagtagaacaactggccacatacacttactcacataaggatcaggcggcagccatttcaagcgggtcgtttgataccgatggaatgattgttgcgccgtgcagcatgaaatctctcgcaagcattcgcacaggaatggcggataatctgctgacacgtgcggcggatgtcatgctcaaggagagaaaaaaactcgtcctcttaacgagagagacgcctttgaaccaaattcatctcgaaaatatgctagcgcttacgaaaatgggcaccatcattcttcctccgatgccggcattttataatcggccgagaagcttagaggaaatggttgaccatattgtttttagaacgttggaccaattcggcattcggcttcctgaagcgaagcgctggaatgggattgaaaaacaaaaaggaggagcttga

SEQ ID NO: 2 - protein, 204 aa, Bacillus subtilis
MKAEFKRKGGGKVKLVVGMTGATGAIFGVRLLQWLKAAGVETHLVVSPWANVTIKHETGYTLQEVEQLATYTYSHKDQAAAISSGSFDTDGMIVAPCSMKSLASIRTGMADNLLTRAADVMLKERKKLVLLTRETPLNQIHLENMLALTKMGTIILPPMPAFYNRPRSLEEMVDHIVFRTLDQFGIRLPEAKRWNGIEKQKGGA

SEQ ID NO: 3 - DNA, 1422 bp, Bacillus subtilis
atggcttatcaagatttcagagaatttctcgctgcccttgaaaaagaaggacagctgcttacagtgaatgaagaggtaaagccggaaccggatttaggggcctccgcacgggcagccagcaatcttggcgataaaagccctgcgctcttatttaacaacatttacggctatcataacgcgcgaattgcgatgaatgtcatcggctcttggccaaaccatgccatgatgctgggcatgccgaaagacacaccggtaaaagaacagttttttgaattcgcaaagcgttatgaccagtttccgatgccggtcaaacgtgaggaaacagcgccatttcatgaaaatgaaatcacagaagatatcaatttgttcgatatactgcctcttttcagaattaaccagggtgatggaggctactatttagacaaagcatgtgtcatttcccgtgatcttgaggaccctgacaacttcggcaaacaaaatgtcggcatttacagaatgcaagtcaaaggaaaagaccgccttggcattcagcctgtcccgcagcacgatattgcaatccatctgcgccaagctgaagaacgcggcatcaaccttccggtcactattgcgctcggctgtgagccggtcattacaacggcggcatcgactccgcttctctatgatcaatcagaatacgaaatggcaggtgcgattcaaggcgaaccatatcgcatcgtcaaatcaaagctgtctgatcttgatgttccgtggggcgctgaagtggtgcttgaaggtgagattattgccggagagcgcgaatatgaagggccgttcggtgaattcacaggccattattccggcggacgcagcatgccgattatcaaaattaaacgcgtctatcacagaaacaatccgatctttgaacatttatacttaggcatgccttggacagaatgcgattacatgatcggcattaacacatgcgtgccgctttatcagcagttaaaagaagcgtatccgaacgaaattgtggcagtgaacgccatgtacacacacggtttaatcgcgattgtttccacaaaaacccgctatggcggatttgcgaaagcggtcggcatgcgcgcactcacaacgccgcacggactcggctactgcaaaatggtcatagtcgttgatgaggatgtcgatccattcaaccttccgcaggtcatgtgggcgctttcgaccaaaatgcatccgaaacatgatgcggtcatcattccggacttatctgtcctgccgcttgatccgggatccaatccatcaggaatcactcacaaaatgattctcgacgccactacaccggttgcgccggaaacaagaggccattattcacagccgcttgattctccgctaacaacgaaagaatgggaacaaaaactaatggacttaatgaataaataa

SEQ ID NO: 4 - protein, 473 aa, Bacillus subtilis
MAYQDFREFLAALEKEGQLLTVNEEVKPEPDLGASARAASNLGDKSPALLFNNIYGYHNARIAMNVIGSWPNHAMMLGMPKDTPVKEQFFEFAKRYDQFPMPVKREETAPFHENEITEDINLFDILPLFRINQGDGGYYLDKACVISRDLEDPDNFGKQNVGIYRMQVKGKDRLGIQPVPQHDIAIHLRQAEERGINLPVTIALGCEPVITTAASTPLLYDQSEYEMAGAIQGEPYRIVKSKLSDLDVPWGAEVVLEGEIIAGEREYEGPFGEFTGHYSGGRSMPIIKIKRVYHRNNPIFEHLYLGMPWTECDYMIGINTCVPLYQQLKEAYPNEIVAVNAMYTHGLIAIVSTKTRYGGFAKAVGMRALTTPHGLGYCKMVIVVDEDVDPFNLPQVMWALSTKMHPKHDAVIIPDLSVLPLDPGSNPSGITHKMILDATTPVAPETRGHYSQPLDSPLTTKEWEQKLMDLMNK

SEQ ID NO: 5 - DNA, 228 bp, Bacillus subtilis
atgcatacatgtcctcgatgcgactcaaaaaagggagaagtcatgagcaaatcgcctgtagaaggcgcatgggaagtttatcagtgccaaacatgcttttttacatggagatcctgtgaaccggaaagcattacaaatcccgaaaaatacaatccagcgtttaaaattgatccaaaggaaacagaaacagcaattgaagttccggcggtgccggaacgaaaggcttga

SEQ ID NO: 6 - protein, 75 aa, Bacillus subtilis
MHTCPRCDSKKGEVMSKSPVEGAWEVYQCQTCFFTWRSCEPESITNPEKYNPAFKIDPKETETAIEVPAVPERKA

SEQ ID NO: 7 - DNA, 1107 bp, Saccharomyces cerevisiae
atgaaaataaaaattttagtagatgaatataatacgcagaaagaacaggctaaaaaagtacgaaaagcaacttcattatatttccgcattcatccttcaattatggccagcaagactttgagggttctttttctgggtcccaaaggtacgtattcccatcaagctgcattacaacaatttcaatcaacatctgatgttgagtacctcccagcagcctctatcccccaatgttttaaccaattggagaacgacactagtatagattattcagtggtaccgttggaaaattccaccaatggacaagtagttttttcctatgatctcttgcgtgataggatgatcaaaaaagccctatccttacctgctccagcagatactaatagaattacaccagatatagaagttatagcggagcaatatgtacccattacccattgtctaatcagcccaatccaactaccaaatggtattgcatcccttggaaattttgaagaagtcataatacactcacatccgcaagtatggggccaggttgaatgttacttaaggtccatggcagaaaaatttccgcaggtcacctttataagattggattgttcttccacatctgaatcagtgaaccaatgcattcggtcatcaacggccgattgcgacaacattctgcatttagccattgctagtgaaacagctgcccaattgcataaggcgtacatcattgaacattcgataaatgataagctaggaaatacaacaagatttttagtattgaagagaagggagaacgcaggcgacaatgaagtagaagacactggattactacgggttaacctactcacctttactactcgtcaagatgaccctggttctttggtagatgttttgaacatactaaaaatccattcactcaacatgtgttctataaactctagaccattccatttggacgaacatgatagaaactggcgatatttatttttcattgaatattacaccgagaagaataccccaaagaataaagaaaaattctatgaagatatcagcgacaaaagtaaacagtggtgcctgtggggtacattccccagaaatgagagatattatcacaaataa

SEQ ID NO: 8 - protein, 368 aa, Saccharomyces cerevisiae
MKIKILVDEYNTQKEQAKKVRKATSLYFRIHPSIMASKTLRVLFLGPKGTYSHQAALQQFQSTSDVEYLPAASIPQCFNQLENDTSIDYSVVPLENSTNGQVVFSYDLLRDRMIKKALSLPAPADTNRITPDIEVIAEQYVPITHCLISPIQLPNGIASLGNFEEVIIHSHPQVWGQVECYLRSMAEKFPQVTFIRLDCSSTSESVNQCIRSSTADCDNILHLAIASETAAQLHKAYIIEHSINDKLGNTTRFLVLKRRENAGDNEVEDTGLLRVNLLTFTTRQDDPGSLVDVLNILKIHSLNMCSINSRPFHLDEHDRNWRYLFFIEYYTEKNTPKNKEKFYEDISDKSKQWCLWGTFPRNERYYHK

SEQ ID NO: 9 - DNA, 1125 bp, Saccharomyces cerevisiae
atgtcactaatggacaattggaagactgatatggaaagttacgatgaaggaggcctagttgctaatccgaacttcgaggttctggccactttcaggtacgaccctggttttgcacgccagtcagcgtcaaagaaagagatctttgaaactccagaccctcgattaggtttgagagacgaagatattaggcagcagataattaatgaggattactcaagttatttacgagtaagggaggttaattccggcggtgaccttctcgaaaatattcagcatcctgatgcttggaagcatgattgcaagaccattgtgtgccagcgtgtagaagatatgctacaagtcatttatgaacgattttttttattagatgaacaataccaaagaataagaatagcattatcatactttaaaattgacttcagcacgtctctgaatgatttattgaagttattggttgaaaacttgattaattgtaaagaaggaaattcagagtatcacgaaaaaattcaaaaaatgatcaacgaaaggcaatgctataaaatgcgggtacttgtctctaagacaggagatatacgaattgaggcaattccaatgcctatggagcctatcctaaaattaacaaccgattatgacagtgtttccacatacttcatcaaaacgatgctcaatggatttttaattgatagcacaataaattgggatgttgttgtttcatctgaaccattgaacgcatcagctttcaccagttttaaaaccacttcaagagatcattacgctagggcgagagttcgcatgcaaactgctataaataacttaagaggttcagaacctacttcttctgtctcgcaatgcgaaattttattttccaacaaatctggcctgctgatggaaggttcaataacaaacgtggctgtaattcaaaaagatcctaacggttctaaaaagtatgtgacaccaagattagcaactggatgtttgtgcggaacaatgcgtcattatttattgcggctcggccttattgaagagggagatatagatataggaagccttaccgttggcaacgaagttttgcttttcaatggcgtcatgggatgcataaagggaacagtgaagacaaaatattga

SEQ ID NO: 10 - protein, 374 aa, Saccharomyces cerevisiae
MSLMDNWKTDMESYDEGGLVANPNFEVLATFRYDPGFARQSASKKEIFETPDPRLGLRDEDIRQQIINEDYSSYLRVREVNSGGDLLENIQHPDAWKHDCKTIVCQRVEDMLQVIYERFFLLDEQYQRIRIALSYFKIDFSTSLNDLLKLLVENLINCKEGNSEYHEKIQKMINERQCYKMRVLVSKTGDIRIEAIPMPMEPILKLTTDYDSVSTYFIKTMLNGFLIDSTINWDVVVSSEPLNASAFTSFKTTSRDHYARARVRMQTAINNLRGSEPTSSVSQCEILFSNKSGLLMEGSITNVAVIQKDPNGSKKYVTPRLATGCLCGTMRHYLLRLGLIEEGDIDIGSLTVGNEVLLFNGVMGCIKGTVKTKY

SEQ ID NO: 11 - DNA, 1560 bp, Saccharomyces cerevisiae
atgttcagtagatctacgctctgcttaaagacgtctgcatcctccattgggagacttcaattgagatatttctcacaccttcctatgacagtgcctatcaagctgcccaatgggttggaatatgagcaaccaacggggttgttcatcaacaacaagtttgttccttctaaacagaacaagaccttcgaagtcattaacccttccacggaagaagaaatatgtcatatttatgaaggtagagaggacgatgtggaagaggccgtgcaggccgccgaccgtgccttctctaatgggtcttggaacggtatcgaccctattgacaggggtaaggctttgtacaggttagccgaattaattgaacaggacaaggatgtcattgcttccatcgagactttggataacggtaaagctatctcttcctcgagaggagatgttgatttagtcatcaactatttgaaatcttctgctggctttgctgataaaattgatggtagaatgattgatactggtagaacccatttttcttacactaagagacagcctttgggtgtttgtgggcagattattccttggaatttcccactgttgatgtgggcctggaagattgcccctgctttggtcaccggtaacaccgtcgtgttgaagactgccgaatccaccccattgtccgctttgtatgtgtctaaatacatcccacaggcgggtattccacctggtgtgatcaacattgtatccgggtttggtaagattgtgggtgaggccattacaaaccatccaaaaatcaaaaaggttgccttcacagggtccacggctacgggtagacacatttaccagtccgcagccgcaggcttgaaaaaagtgactttggagctgggtggtaaatcaccaaacattgtcttcgcggacgccgagttgaaaaaagccgtgcaaaacattatccttggtatctactacaattctggtgaggtctgttgtgcgggttcaagggtgtatgttgaagaatctatttacgacaaattcattgaagagttcaaagccgcttctgaatccatcaaggtgggcgacccattcgatgaatctactttccaaggtgcacaaacctctcaaatgcaactaaacaaaatcttgaaatacgttgacattggtaagaatgaaggtgctactttgattaccggtggtgaaagattaggtagcaagggttacttcattaagccaactgtctttggtgacgttaaggaagacatgagaattgtcaaagaggaaatctttggccctgttgtcactgtaaccaaattcaaatctgccgacgaagtcattaacatggcgaacgattctgaatacgggttggctgctggtattcacacctctaatattaataccgccttaaaagtggctgatagagttaatgcgggtacggtctggataaacacttataacgatttccaccacgcagttcctttcggtgggttcaatgcatctggtttgggcagggaaatgtctgttgatgctttacaaaactacttgcaagttaaagcggtccgtgccaaattggacgagtaa

SEQ ID NO: 12 - protein, 519 aa, Saccharomyces cerevisiae
MFSRSTLCLKTSASSIGRLQLRYFSHLPMTVPIKLPNGLEYEQPTGLFINNKFVPSKQNKTFEVINPSTEEEICHIYEGREDDVEEAVQAADRAFSNGSWNGIDPIDRGKALYRLAELIEQDKDVIASIETLDNGKAISSSRGDVDLVINYLKSSAGFADKIDGRMIDTGRTHFSYTKRQPLGVCGQIIPWNFPLLMWAWKIAPALVTGNTVVLKTAESTPLSALYVSKYIPQAGIPPGVINIVSGFGKIVGEAITNHPKIKKVAFTGSTATGRHIYQSAAAGLKKVTLELGGKSPNIVFADAELKKAVQNIILGIYYNSGEVCCAGSRVYVEESIYDKFIEEFKAASESIKVGDPFDESTFQGAQTSQMQLNKILKYVDIGKNEGATLITGGERLGSKGYFIKPTVFGDVKEDMRIVKEEIFGPVVTVTKFKSADEVINMANDSEYGLAAGIHTSNINTALKVADRVNAGTVWINTYNDFHHAVPFGGFNASGLGREMSVDALQNYLQVKAVRAKLDE

SEQ ID NO: 13 - DNA, 29 bp, Artificial Sequence
  Synthetic (forward primer 1)
aaacatatgaaaataaaaattttagtaga

SEQ ID NO: 14 - DNA, 29 bp, Artificial Sequence
  Synthetic (reverse primer 1)
aaactcgagtttgtgataatatctctcat

SEQ ID NO: 15 - DNA, 29 bp, Artificial Sequence
  Synthetic (forward primer 2)
aaacatatgaaagcagaattcaagcgtaa

SEQ ID NO: 16 - DNA, 29 bp, Artificial Sequence
  Synthetic (reverse primer 2)
aaactcgagagcctttcgttccggcaccg

SEQ ID NO: 17 - DNA, 29 bp, Artificial Sequence
  Synthetic (forward primer 3)
aaacatatgtcactaatggacaattggaa

SEQ ID NO: 18 - DNA, 29 bp, Artificial Sequence
  Synthetic (reverse primer 3)
aaactcgagatattttgtcttcactgttc

(19) United States
(12) Patent Application Publication
(10) Pub. No.: US 2014/0155570 A1
(43) Pub. Date: Jun. 5, 2014

(54) PROCESS OF BIOLOGICALLY PRODUCING TEREPHTHALIC ACID AND DERIVATIVE THEREOF

(71) Applicant: Samsung Electronics Co., Ltd., Suwon-si (KR)

(72) Inventors: Won Jae CHOI, Seongnam-si (KR); Jin Ho AHN, Seongnam-si (KR); Jong Won BYUN, Suwon-si (KR); Young Wan HA, Seoul (KR)

(73) Assignee: Samsung Electronics Co., Ltd., Suwon-si (KR)

(21) Appl. No.: 14/091,478

(22) Filed: Nov. 27, 2013

(30) Foreign Application Priority Data
Nov. 30, 2012 (KR) 10-2012-0138374

(51) Int. Cl. C12P 7/44; C08G 63/00

(52) U.S. Cl. CPC C12P 7/44; C08G 63/00
USPC 528/308.6; 435/142

(57) ABSTRACT

A method of biologically producing an terephthalic acid or a derivative thereof by contacting a substrate containing an aromatic carboxylic acid with a biocatalyst that adds a carboxyl group at the para-position of the aromatic carboxylic acid.

### CROSS-REFERENCE TO RELATED APPLICATIONS

This application claims the benefit of Korean Patent Application No. 10-2012-0138374, filed on Nov. 30, 2012, in the Korean Intellectual Property Office, the entire disclosure of which is incorporated herein by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 27,600 Byte ASCII (Text) file named “715454_ST25.TXT,” created on Nov. 19, 2013.

### BACKGROUND OF THE INVENTION

1. Field of the Invention

The present invention relates to the biological production of terephthalic acid and its derivatives. More particularly, the present invention relates to a process of biologically producing terephthalic acid and its derivatives by degrading lignin through chemical or biological conversion and biologically converting the lignin breakdown product into terephthalic acid or derivatives thereof.

2. Description of the Related Art

A terephthalic acid is a useful starting material for a wide range of applications, such as a polyester fiber of polyethylene terephthalate (PET) and polytrimethylene terephthalate (PTT), a packing material and bottle material. Particularly, PET has been widely used as a packing material due to its transparence, mechanical strength, and gas barrier capacity. The terephthalic acid as a raw material preparing for PET is prepared at an amount of 50,000,000 tons every year, and 100,000 tons to 800,000 tons in a single plant every year.

PET is synthetic polymer consisting of terephthalic acid and mono ethylene glycol, both derived from petroleum. Specifically terephthalic acid is industrially manufactured from p-xylene via catalytic oxidation. Unexpected increases in crude oil prices results in high manufacturing costs for aromatic raw materials such as benzene, toluene, and xylene, indicating that terephthalic acid production is very much dependent on crude oil price. Furthermore, conventional petroleum-based terephthalic acid production is recognized as nonrenewable and generates a lot of greenhouse gases. In this context, there is an increasing demand for alternative route for terephthalic acid production via industrial biotechnology using renewable biomass as feedstock.

Terephthalic acid is prepared by gaseous oxidation from para-xylene. More specifically, crude terephthalic acid is prepared by the oxidation with aerating the para-xylene in acetic acid solvent in the presence of catalyst such as cobalt, manganese or bromide, and is dissolved in water and reduced by hydrogen to produce the purified terephthalic acid. This method is referred to as the Amoco MC method.

However, the process of chemically synthesizing terephthalic acid has various serious problems, namely dangerous working conditions requiring high temperature and pressure and environmental contamination caused by the complicated separation and purification steps due to by-products and chemical wastes produced in the separation and purification steps.

Bromide is used as a reaction accelerating agent. Although the bromide functions as the initiation and acceleration of oxidation, it causes device corrosion, and therefore, results in a periodic need to replace the production facilities. Furthermore, bromide is very toxic to human body. When a very small amount of bromide is inhaled or contacted, it is very lethal to humans, worsens the working environment, and causes air contamination.

Compared with other conventional reactions, the Amoco MC method uses an acetic acid as a reaction solvent. Acetic acid helps the catalytic action of the reaction by increasing the complex formation between a heavy metal catalyst and a reaction accelerating agent, but has some disadvantages. That is, when acetic acid is used at a high temperature, it needs an expensive anti-corrosive material used in the reactor and its surrounding equipment due to corrosive capacity, thereby increasing the production cost. Like bromide, acetic acid is very harmful to humans.

Accordingly, some methods for an environmentally-friendly, simple, and cost effective preparation of terephthalic acid have been developed, including a process of biologically preparing terephthalic acid from petroleum-based feedstock.

Among the biological process using fossil fuel, terephthalic acid has been prepared from hexadecane and p-xylene by using Nocardia culture (SU419509). Both methyl groups of p-xylene were oxidized simultaneously by using Burkholderia culture (U.S. Pat. No. 6,461,840B1). However, these methods do not represent truely environmentally-friendly biological processes, because they still use fossil fuel as a substrate.

US2011/0207185A1 discloses the preparation of terephthalic acid by using a recombinant microorganism having a (2-hydroxy-3-methyl-4-oxobutoxy)phosphonate, p-toluate, or terephthalate pathway. However, the method requires glucose as a substrate, which is mainly derived from edible resources.

There is therefore impertinent strong need for a biological process of preparing terephthalic acid from a renewable biomass.

### SUMMARY OF THE INVENTION

An embodiment of the present invention provides a biological process of producing terephthalic acid or its derivative, which is of higher specificity, compared to chemical processes.

Another embodiment provides a biological process of producing a terephthalic acid and its derivative in an environment-friendly manner from a lignin degradation product which is degraded economically and effectively from lignin.

A further embodiment pertains to a biological process of producing terephthalic acid or a derivative thereof. More particularly, terephthalic acid or a derivative thereof can be biologically prepared from an aromatic carboxylic acid or a derivative thereof.

Still further embodiment provides the production of terephthalic acid or a derivative thereof from lignin of a recyclable biomass. In this regard, lignin is chemically and/or biologically degraded to give lignin breakdown products including aromatic monomers, for example benzoic acid followed by biological conversion of the breakdown products into terephthalic acid or a derivative thereof.

Focusing on the synthesis of terephthalic acid or a derivative thereof from non-edible biomass, the present invention is configured to chemically or biologically degrade lignin, an inexpensive raw material, into aromatic monomers and to chemically or biologically convert the aromatic monomers into terephthalic acid, thus eliminating dependence on petrochemical materials.

By using a biological process in which a substrate including an aromatic carboxylic acid having a p-hydroxyl group, or a derivative thereof is contacted with a biocatalyst having an activity to introducing p-carboxyl group, an embodiment of the present invention allows terephthalic acid or a derivative thereof to be produced in an environment-friendly manner and at higher specificity, compared to a chemical process.

An embodiment provides the production of terephthalic acid or a derivative thereof from lignin. In this regard, lignin may be degraded to give lignin breakdown products including aromatic monomers, followed by biological conversion of the breakdown products into terephthalic acid or a derivative thereof.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows HPLC chromatograms of a standard solution of enzyme reaction intermediates comprising p-hydroxybenzoic acid, benzoic acid and terephthalic acid, as quantitatively measured by Waters e2695 HPLC and Waters 2489 UV/VIS (253 nm, 280 nm) detector.

FIGS. 2A and 2B shows mass spectra of the produced terephthalic acid obtained by the enzyme reaction (FIG. 2A) and the standard solution of terephthalic acid (FIG. 2B).

### DESCRIPTION OF THE PREFERRED EMBODIMENTS

An embodiment of the present invention addresses a process of biologically producing an aromatic carboxylic acid represented by the following Chemical Formula 1 or a derivative thereof, including contacting a substrate including an aromatic carboxylic acid represented by the following Chemical Formula 2, with a biocatalyst having a catalytic activity to introducing a carboxyl group at p-position, as illustrated in the following Reaction Scheme 1.

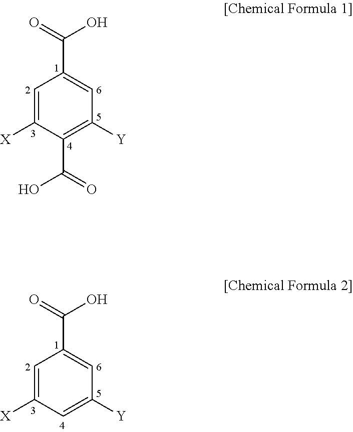

wherein, X and Y, which may be the same or different, are independently hydrogen, hydroxy, or C1-C4 alkoxy. The C1-C4 alkoxy may be linear or branched, and is preferably methoxy or ethoxy.

The exemplified aromatic carboxylic acid represented by the following Chemical Formula 1 or an derivative thereof are a terephthanlic acid, m-hydroxy terephthanlic acid, 3,5-dihydroxy terephthalic acid, 3-methoxy terephthalic acid, 3,5-dimethoxy terephthanlic acid, and the like.

The compound of Chemical Formula 2 corresponds to the compound of Chemical Formula 1 and can be obtained by the petrochemical method or biological method. For example, the compound of Chemical Formula 2 may be obtained by the industrial chemical process including a partial oxidation of toluene or the biological method using a microorganism, such as S. maritimus and the like (Shuhei et. al., Microbial Cell Factories 2012, 11:49). Alternatively, the compound of Chemical Formula 2 can be obtained from the compound of Chemical Formula 3. The examples of the compound represented by Chemical Formula 2 include benzoic acid, m-hydroxybenzoic acid, 3,5-dihydroxybenzoic acid, 3-methoxybenzoic acid, 3,5-dimethoxybenzoic acid, and the like.

The aromatic carboxylic acid represented by Chemical Formula 2 may be prepared by contacting the substrate including the aromatic carboxylic acid represented by Chemical Formula 3, with a biocatalyst having an activity to remove the p-hydroxy group of Chemical Formula 3. More specifically, before or simultaneously contacting with the biocatalyst having an activity to introduce the carboxyl group to p-position, the method of producing the compound represented by Chemical Formula 1 further includes a step of producing an aromatic carboxylic acid represented by Chemical Formula 2 which is prepared by contacting the substrate including the aromatic carboxylic acid represented by Chemical Formula 3, with a biocatalyst having an activity to remove the p-hydroxyl group of Chemical Formula 3.

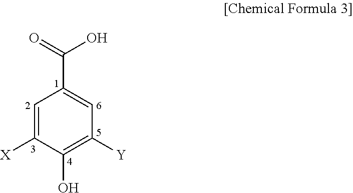

wherein, X and Y, which may be the same or different, are independently hydrogen, hydroxyl, or C1-C4 alkoxy. The C1-C4 alkoxy may be linear or branched, with preference for methoxy or ethoxy.

The compound of Chemical Formula 3 which corresponds to the compound of Chemical Formula 2, may be produced by using a petrochemical process or a biological process. For example, it may be produced using a commercial chemical process based on the Kolbe-Schmitt reaction, or through an aromatic amino acid biosynthesis pathway (Edwin Ritzer and Rudolf Sundermann “Hydroxycarboxylic Acids, Aromatic” in Ullmann's Encyclopedia of Industrial Chemistry 2002, Wiley-VCH, Weinheim; Biotechnol Bioeng. 2001 December; 76(4):376-90). Alternatively, the compound of Chemical Formula 3 may be obtained from a breakdown product of lignin after chemical and/or biological degradation, or from a compound of Chemical Formula 4, as will be further explained. The degradation of lignin may be carried out using at least one selected from the group consisting of pyrolysis, gasification, hydrogenolysis, acidolysis, alkaline lysis, chemical oxidation, hydrolysis under supercritical conditions, and enzymolysis. Preferred examples of the compound of Chemical Formula 2 include 4-hydroxybenzoic acid, 3,4-dihydroxybenzoic acid, 4,5-dihydroxybenzoic acid, 3,4,5-trihydroxybenzoic acid, 3-methoxy-4-hydroxybenzoic acid, 4-hydroxy-5-methoxybenzoic acid, and 3,5-dimethoxy-4-hydroxybenzoic acid.

In one embodiment, the aromatic carboxylic acid having a p-hydroxy group of Chemical Formula 3 may be prepared from an aromatic aldehyde having a p-hydroxyl group of Chemical Formula 4 by chemical oxidation or biocatalytic oxidation.

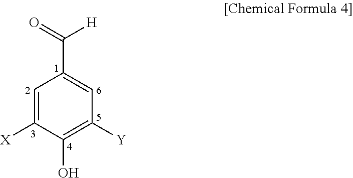

wherein, X and Y, which may be the same or different, are independently hydrogen, hydroxy, or C1-C4 alkoxy. The C1-C4 alkoxy may be linear or branched, with preference for methoxy or ethoxy. Examples of the compound of Chemical Formula 4 are p-hydroxybenzaldehyde, vanillin, and syringaldehyde.

The compound of Chemical Formula 4, which corresponds to the compound of Chemical Formula 3, may be produced using a petrochemical process or a biological process. For example, it may be obtained by chemically or biologically degrading lignin. In one embodiment, the process of the present invention may further include degrading lignin to provide a breakdown product of lignin including the aromatic aldehyde having a p-hydroxyl group of Chemical Formula 4; and oxidizing the breakdown product of lignin to convert the aromatic aldehyde having a p-hydroxy group into an aromatic carboxylic acid having p-hydroxyl group of Chemical Formula 3.

A process of producing a terephthalic acid or a derivative thereof from an aromatic aldehyde in accordance with an embodiment of the present invention is illustrated in Reaction Scheme 1. Specifically, the aromatic aldehyde having a p-hydroxy group of Chemical Formula 4 is chemically or biologically oxidized into an aromatic carboxylic acid having a p-hydroxy group of Chemical Formula 3, followed by biological removal of the p-hydroxy group to give a p-hydroxy-free aromatic carboxylic acid represented by Chemical Formula 2, and then introducing a carboxylic acid at p-position to produce a terephthalic acid or a derivative thereof represented by Chemical Formula 1.

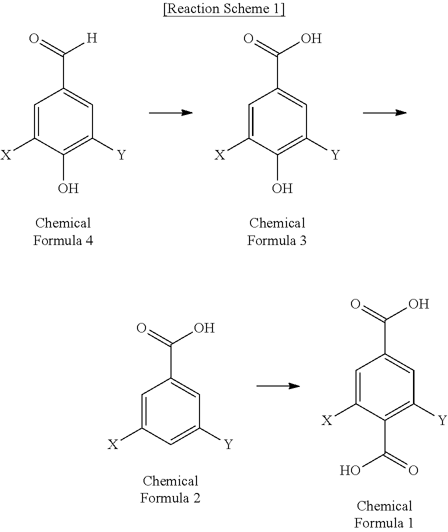

When X and Y in the compounds of Chemical Formulae 1 to 4 are not simultaneously a hydrogen substituent, the process of the present invention may further comprise removing at least one substituent selected from the group consisting of hydroxy and C1-C4 alkoxy at either or both positions 3′ and 5′ of the benzene ring. The removal of the substituent may be carried out prior to, simultaneously with, or after each step of Reaction Scheme 1.

According to one embodiment, when X and Y are not simultaneously hydrogen in Chemical Formula 2, the process of the present invention may further comprises contacting the substrate with a biocatalyst having the catalytic activity to remove a substituent selected from the group consisting of hydroxy and C1-C4 alkoxy at either or both of positions 3 and 5 on the benzene ring before, simultaneously with, or after contacting with the biocatalyst having the catalytic activity to introducing a carboxyl group at p-position. For example, when X and Y in Chemical Formula 2 are hydrogen and methoxy, respectively, the introduction of carboxyl group at p-position and the removal of methoxy of Y may give a terephthalic acid.

According to one embodiment, when X and Y are not simultaneously hydrogen in Chemical Formula 3, the process of the present invention may further include contacting the substrate with a biocatalyst having the catalytic activity to remove a substituent selected from the group consisting of hydroxyl group and C1-C4 alkoxy at either or both of positions 3 and 5 on the benzene ring before, simultaneously with, or after contacting with the biocatalyst having the catalytic activity to remove a hydroxyl group at p-position.

According to another embodiment, when X and Y are not simultaneously hydrogen in Chemical Formula 4, the process of the present invention may further include contacting the substrate with a biocatalyst having the catalytic activity to remove a substituent selected from the group consisting of hydroxy and C1-C4 alkoxy at either or both of positions 3 and 5 on the benzene ring before, simultaneously with, or after the oxidation.

The process illustrated in Reaction Scheme 1, involves a biocatalyst having a catalytic activity to introduce p-carboxyl group to the aromatic carboxylic acid by contacting with a substrate including an aromatic carboxylic acid of Chemical Formula 2, a biocatalyst having a catalytic activity to remove p-hydroxy from an aromatic carboxylic acid having a p-hydroxy group of Chemical Formula 3, and a biocatalyst having a catalytic activity to remove at least one substituent selected from the group consisting of hydroxy and C1-C4 alkoxy at either or both 3- and 5-positions on the aromatic carboxylic acid.

Any enzyme may be used in the present invention as a biocatalyst having a catalytic activity to introduce p-carboxyl group to the aromatic carboxylic acid, so long as it can introduce a carboxylic group at p-position of the benzene ring from the aromatic carboxylic acid or its derivatives. The examples of biocatalyst having a catalytic activity to introduce p-carboxyl group to the aromatic carboxylic acid include at least one selected from the group consisting of aminobenzoate decarboxylase (EC 4.1.1.24), 3-Hydroxy-2-methylpyridine-4,5-dicarboxylate 4-decarboxylase (EC 4.1.1.51), 4,5-Dihydroxyphthalate decarboxylase (EC 4.1.1.55), Orsellinate decarboxylase (EC 4.1.1.58) (Eur. J. Biochem, 1971, 26:22(4); 485-8), Gallate decarboxylase (EC 4.1.1.59) (J. Bacteriol. 1987, 169(5): 1886-90), 4-hydroxybenzoate decarboxylase (EC 4.1.1.61), Protocatechuate decarboxylase (EC 4.1.1.63), 3,4-dihydroxyphthalate decarboxylase (EC 4.1.1.69), phenylphosphate carboxylase (EC 4.1.1.x) (J. Bacteriol. 2004 July; 186(14):4556-67). 4-hydroxybenzoate decarboxylase, for examples the enzymes including an amino acid sequence as set forth in SEQ ID NOs: 2, 4 or 6 or an amino acid sequence encoded by the nucleotide sequence as set forth in SEQ ID NOs: 1, 3 or 5 can be preferably used Specific examples of enzymes include Gene Ontology Number (GO No.) GO:0018799, GO:0047662, GO:0050223, GO:0050159, GO:0018798, GO:0018796, GO:0047556, GO:0047431, GO:0018862 and the like.

The enzymes are summarized together with their genes in Table 1.

TABLE 1

| | | GenBank | |
|---|---|---|---|
| Enzyme | Gene | Accession No. | Microbial Source |
| Aminobenzoate | hpaH | ACO02919.1 | Brucella melitensis |
| decarboxylase | | | |
| 3-Hydroxy-2- | mlr6791 | BAB53020.1 | Rhizobium loti (strain AFF303099) |
| methylpyridine-4,5- | | | |
| dicarboxylate 4- | | | |
| decarboxylase | | | |
| 4,5-Dihydroxyphthalate | phtD | BAA03974.1 | Comamonas testosterone |
| decarboxylase | | | |
| 4-hydroxybenzoate | RB223 | CAD71454.1 | Rhodopirellula baltica |
| decarboxylase | | | |
| Protocatechuate | aroY | BAH20873.1 | Klebsiella pneumoniae subsp. |
| decarboxylase | | | Pneumoniae |
| 3,4-dihydroxyphthalate | phtC | AAK16538.1 | Arthrobacter keyseri |
| decarboxylase | | | |
| phenylphosphate | ppcA | CAI07883.1 | Aromatoleum aromaticum |
| carboxylase | | | |

As long as a biocatalyst is capable of removing the hydroxy group at para-position of the compound of Chemical Formula 3 to provide the compound of Chemical Formula 2, any biocatalyst can be used for the present invention. The examples of the biocatalyst include at least one selected from the group consisting of bile-acid 7-alpha-dehydroxylase (EC 1.17.99.5), 4-hydroxybenzoyl-CoA reductase (EC 1.3.7.9), 3-dehydroquinate hydro-lyase (EC 4.2.1.10), aldos-2-ulose dehydratase (EC 4.2.1.110), Biochim Biophys Acta. 2005, 1723(1-3):63-73), o-succinylbenzoate synthase (EC 4.2.1.113), 3-dehydroshikimate hydro-lyase (EC 4.2.1.118), prephenate hydro-lyase (EC 4.2.1.51), arogenate dehydratase (EC 4.2.1.91), scytalone 7,8-hydro-lyase (EC 4.2.1.94), and 16α-hydroxyprogesterone hydro-lyase (EC 4.2.1.98) (J Steroid Biochem Mol. Biol. 1991, 38(2):257-63), but not limited thereto. The particular example of the biocatalyst may be a prephenate hydro-lyase having an amino acid sequence (PHA2) as set forth in SEQ ID NO: 8 or an amino acid sequence encoded by the nucleotide sequence as set forth in SEQ ID NO: 7.

The specific examples of the enzymes includes Gene Ontology Number (GO No.) GO:0047769, GO:0004664, GO:0046565, GO:0003855, GO:0030411, GO:0033991, GO:0043748, GO:0047455, GO:0018525, GO:0033792 and the like.

The enzymes are summarized together with their genes in Table 2.

TABLE 2

| | | GenBank | |
|---|---|---|---|
| Enzyme | Gene | Accession No. | Microbial Source |
| bile-acid 7-alpha- | baiA2 | AAB61150.1 | Eubacterium sp. (strain VPI 12708) |
| dehydroxylase | | | |
| 4-hydroxybenzoyl-CoA | hcrA | CAA05038.1 | Thauera aromatic |
| reductase | | | |
| 3-dehydroquinate hydro- | aroD | ACR61804.1 | Escherichia coli |
| lyase | | | |
| o-succinylbenzoate | menC | AAA71917.1 | Escherichia coli |
| synthase | | | |
| 3-dehydroshikimate hydro- | qa-4 | CAA32750.1 | Neurospora crassa |
| lyase | | | |
| prephenate hydro-lyase | PHA2 | DAA10245.1 | Saccharomyces cerevisiae |
| arogenate dehydratase | Bphy | ACC72194.1 | Burkholderia phymatum |
| scytalone 7,8-hydro-lyase | SDH1 | BAA34046.1 | Magnaporthe oryzae |

For instance, prephenate hydro-lyase (PHA2) is capable of removing the hydroxy group at para-position of the compound of Chemical Formula 3 to afford the compound of Chemical Formula 2, which can be converted to the compound of Chemical Formula 1 by 4-hydroxybenzoate decarboxylase.

The catalyst which can remove at least one substituent selected form hydroxy and C1-C4 alkoxy at position 3′ and/or 5′ of the benzene ring from the aromatic carboxylic acid may be an enzyme selected from the group consisting of anthranilate synthase (EC 4.1.3.27), aminodeoxychorismate lyase (EC 4.1.3.38), chorismate lyase (EC 4.1.3.40), 3-dehydroquinate hydro-lyase (EC 4.2.1.10), 3-dehydroshikimate hydro-lyase (EC 4.2.1.118), prephenate hydro-lyase (EC 4.2.1.51), 5-O-(1-carboxyvinyl)-3-phosphoshikimate phosphate-lyase (EC 4.2.3.5), isochorismate lyase (EC 4.2.99.21), and hydroxyphenylpyruvate synthase (EC 5.4.99.5), or a microorganism producing the enzyme, a lysate of the microorganism, or an extract from the microorganism cell lysate. The aminodeoxychorismate lyase (ADC lyase) may have an amino acid sequence as set forth in SEQ ID NO: 10, or an amino acid sequence encoded by the nucleotide sequence as set forth in SEQ ID NO: 9.

Examples of the catalyst having the activity to remove at least one substituent selected form hydroxy and C1-C4 alkoxy at position 3′ and/or 5′ of the benzene ring from the aromatic carboxylic acid include Gene Ontology Numbers (GO Nos.) GO:004049, GO:005950, GO:004107, GO:008813, GO:0046565, GO:0043904, GO:008696, GO:004664, GO:003855, GO:004106 and the like.

The enzymes are summarized, together with their genes in Table 2.

TABLE 3

| | | GenBank | |
|---|---|---|---|
| Enzyme | Gene | Accession No. | Microbial Source |
| Anthranilate synthase | TRP2 | AAA35175.1 | Saccharomyces cerevisiae |
| Aminodeoxychorismate | ABZ2 | DAA10190.1 | Saccharomyces cerevisiae |
| lyase | | | |
| Chorismate lyase | ubiC | CAA47181.1 | Escherichia coli |
| 3-Dehydroquinate hydro- | aroD | ACR61804.1 | Escherichia coli |
| lyase | | | |
| 3-Dehydroshikimate hydro- | quiC | AAC37159.1 | Acinetobacter sp. (strain ADP1) |
| lyase | | | |
| Prephenate hydro-lyase | pheA | AAA22507.1 | Bacillus subtilis |
| 5-O-(1-Carboxyvinyl)-3- | aroC | AAA23487.1 | Escherichia coli |
| phosphoshikimate phosphate | | | |
| lyase | | | |
| Isochorismate lyase | entB | AAA16102.1 | Escherichia coli |
| hydroxyphenylpyruvate | aroH | ADE84133.1 | Rhodobacter capsulatus |
| synthase | | | |

Generally, an enzyme may act on various substrates, and even on unknown substrates. In addition, an enzyme differs in activity from one substrate to another, and can be changed in activity or specificity for a certain substrate through modification, such as mutation or directed evolution. Like this, the enzymes of the present invention can be changed in substrate specificity or enhanced in activity using protein evolution technology so as to increase the productivity of the products.

As mentioned above, the enzymes, microorganisms as enzyme sources, lysates of the microorganisms, or extracts from the microorganism cell lysates may be used as the biocatalyst of the present invention. Contacting the substrate with the biocatalyst may be performed under a condition allowable to produce the product by bringing the substrate into contact with an enzyme, a microorganism containing the enzyme, a lysate of the microorganism, or an extract from the microorganism cell lysate, or culturing the microorganism in a medium containing the substrate.

The enzymatic reaction in each step of the present invention may be achieved by contacting the substrate with a proper enzyme or a microorganism containing the enzyme, or culturing the microorganism in a medium containing the substrate. The enzymatic reaction may be done at a pH of from 5.0 to 10.0, with an optimal pH dependent on the enzyme used. In addition, the enzymatic reaction may be done at a temperature of from 25° C. to 50° C., with the optimal temperature depending on the enzyme employed. In a preferred embodiment of the present invention, conversion from aromatic aldehyde to aromatic carboxylic acid is executed at 30° C.˜37° C.

In a further embodiment of the present invention, the microorganism used in each step of the present invention may be recombinant or wild-type. A recombinant microorganism might be prepared by introducing a gene encoding the enzyme into a host cell using a recombinant technique.

When a recombinant enzyme is used according to one embodiment of the present invention, the process comprises 1) constructing an expression vector carrying a gene coding for the enzyme; 2) transforming the expression vector into a host cell, followed by culturing the host cell; 3) producing the enzyme from the host cell; and 4) reacting the enzyme with the substrate. The enzyme acting on the substrate may be in a pure or crude form.

Any expression vector that is employed in genetic manipulation could be applied to the construction of the recombinant expression vector for use in producing a terephthalic acid or a derivative thereof. So long as it is transformed with the recombinant expression vector to expresses the gene of interest to produce an active enzyme protein, any strain, whether bacterial, fungal, or of yeast, can be used as a host cell in the present invention. Preferred is E. coli.

Focusing on the synthesis of terephthalic acid from biomass, the present invention is configured to chemically or biologically degrade lignin into breakdown products from which aromatic carboxylic acid, particularly terephthalic acid or a derivative thereof is produced.

Within the scope of the lignin of the present invention are lignin, lignin derivatives, lignin fragments, and lignin-containing material. The term “lignin derivatives,” as used herein, is intended to encompass lignin compounds modified by a chemical reaction, such as phenolation, acetylation, etc. The term “lignin fragments” means breakdown products obtained as a result of the chemical or biological degradation of lignin.

Typically, lignin is obtained by separating cellulose and hemicelluloses in the biorefinery or pulping process. There are various types of lignin including kraft lignin (alkaline lignin), dealkaline lignin, hydrolytic lignin, organosolv lignin, and sodium lignin sulfonate, according to production process. As a by-product from the lignocelluloses bioethanol process, lignin can be also used Lignin is an aromatic polymer surrounding microfibers, forming a resinous structure in which phenylpropanoids, such as coumaryl alcohol, coniferyl alcohol, sinapyl alcohol, etc. serve as structural units, being polymerized via carbon-carbon bonds or carbon-oxygen bonds in a haphazard manner.

The degradation of lignin may be biodegradation or physicochemical degradation, the latter being preferred because of higher degradation rate. Biodegradation of lignin may be carried out with enzymes such as peroxidase and laccase. Additionally, lignin may be degraded physicochemically. Among the types of physicochemical degradation available for lignin in the present invention are pyrolysis, gasification, hydrogenolysis, acidolysis, alkaline lysis, chemical oxidation, and hydrolysis under supercritical condition.

In one embodiment, the acidolysis or alkaline lysis of lignin is preferably accomplished by treatment with H₂SO₄, HCl, or HNO₃ at a concentration of 0.1 to 5% (w/v) or with a high concentration (0.5 to 2.0 mol/L) NaOH or KOH solution. Preferably, the acidic or alkaline treatment is carried out at about 80˜350° C. for 5˜120 min.

Turning to pyrolysis, lignin can be degraded at as high as 350˜650° C. using a high pressure reactor. The efficiency of pyrolysis can be increased in the presence of a catalyst such as nitrobenzene, KMnO₄, H₂O₂, zeolite, etc. In addition, the degradation of lignin can be accomplished using other physicochemical methods such as hydrogenolysis and hydrolysis under supercritical conditions.

The degradation of lignin is preferably carried out at an oxygen pressure of 2-20 bar. In addition, the degradation processes are preferably completed within 200 min, but the duration may be adjusted appropriately.

The lignin breakdown products include a mixture of aromatic monomers including aromatic aldehydes, such as vanillin, syringaldehyde, p-hydroxybenzaldehyde, and terephthalic acid such as vanillic acid, syringic acid, p-hydroxybenzoic acid, etc., and contain compounds of Chemical Formula 3 and/or Chemical Formula 4.

In accordance with an aspect thereof; the present invention addresses a process of biologically producing a terephthalic acid represented by Chemical Formula 1, or a derivative thereof; including:

degrading lignin to give a lignin breakdown product including an aromatic carboxylic acid having a p-hydroxyl group of Chemical Formula 3; and

contacting the lignin breakdown product, simultaneously or sequentially with a biocatalyst having an activity to remove a p-hydroxy group from the aromatic carboxylic acid of Chemical Formula 3, and a biocatalyst having an activity to introducing a carboxyl group at p-position of the aromatic carboxylic acid.

When X and Y in Chemical Formula 3 are not simultaneously hydrogen, the method may further include applying to the aromatic carboxylic acid a biocatalyst having an activity to remove at least one substituent selected from hydroxy and C1-C4 alkoxy at either or both positions 3 and 5, before, simultaneously with or after the contact with the biocatalyst having an activity to remove a p-hydroxy group and/or the biocatalyst having an activity to introducing p-carboxyl group.

In addition, when the lignin breakdown product contains an aromatic aldehyde compound of Chemical Formula 4, such as vanillin, syringaldehyde, etc., the process may further include converting the lignin breakdown product into the compound of Chemical Formula 3 by oxidation, prior to or after the contact of the lignin breakdown product including the compound of Chemical Formula 3 with the biocatalyst having an activity to remove p-hydroxy from the compound of Chemical Formula 3.

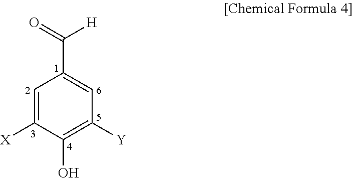

For example, conversion from the aromatic aldehyde of Chemical Formula 4 to the aromatic carboxylic acid of Chemical Formula 3 may be performed by chemical or biological reaction.

For chemical conversion, a silver oxide method or a caustic fusion method may be utilized. First, aromatic monomers with an aldehyde functional group are reacted with 1 M NaOH at 55˜60° C. for about 10 min in the presence of 1 M Ag₂O, followed by neutralization with the equal amount of 1 M HCl with agitation to afford the terephthalic acid as a precipitate.

The biological reaction is performed by using a biocatalyst such as an enzyme, a whole microbial cell, a microbial cell lysate, or a cell extract. The enzyme useful in the present invention may be exemplified by aldehyde dehydrogenase (EC 1.2.1.3, EC 1.2.1.4, EC 1.2.1.5), vanillin dehydrogenase (EC 1.2.1.67) and other enzymes functionally corresponding thereto. Non-limiting examples of these enzymes include GenBank ID CAD60262.1, ABK09332.1, Uniprot ID P47771, and P54114.

The enzymatic reactions in the presence of a pure enzyme as well as a microbial whole cells expressing the enzyme or functionally identical enzymes, such as Saccharomyces cerevisiae, Bacillus subtilis, Escherichia coli, Pseudomonas fluorescens, Pseudomonas putida, Serratia marcescens, Sphingomonas paucimobilis, Streptomyces viridosporus, Desulfovibrio vulgaris, or Burkholderia cepacia, or a lysate or extract thereof are considered as the enzymatic reaction. The aldehyde dehydrogenase may have an amino acid sequence (ALD4) as set forth in SEQ ID NO: 12 or an amino acid sequence encoded by the nucleotide sequence as set forth in SEQ ID NO: 11.

The terephthalic acid can be converted into derivative thereof which is in turn polycondensed with diols to manufacture polyester such as polyethylene terephthalate (hereinafter referred to as “PET”) or polytrimethylene terephthalate (hereinafter referred to as “PTT”). Superior in strength, hygroscopicity, and wrinkle recovery, PET is widely used in synthetic fibers. In addition, PET is used in a variety of packaging products due to its transparency, mechanical properties, and a gas barrier properties. PET is used in applications involving packing foods, soft drinks, alcoholic beverages, detergents, cosmetics, drugs, and edible oils.

By using a biological process in which a substrate including an aromatic carboxylic acid, or a derivative thereof is contacted with a biocatalyst having an activity to introducing p-carboxyl group, the present invention allows terephthalic acid or a derivative thereof to be produced in an environment-friendly manner and at higher specificity, compared to a chemical process. The terephthalic acid or a derivative thereof can be obtained from lignin by degrading the lignin to give lignin breakdown products including aromatic monomers, followed by biological conversion of the breakdown products into terephthalic acid or a derivative thereof.

A better understanding of the present invention may be obtained through the following examples which are set forth to illustrate the present invention, but are not to be construed as limiting the present invention.

<Analysis of Aromatic Monomers>

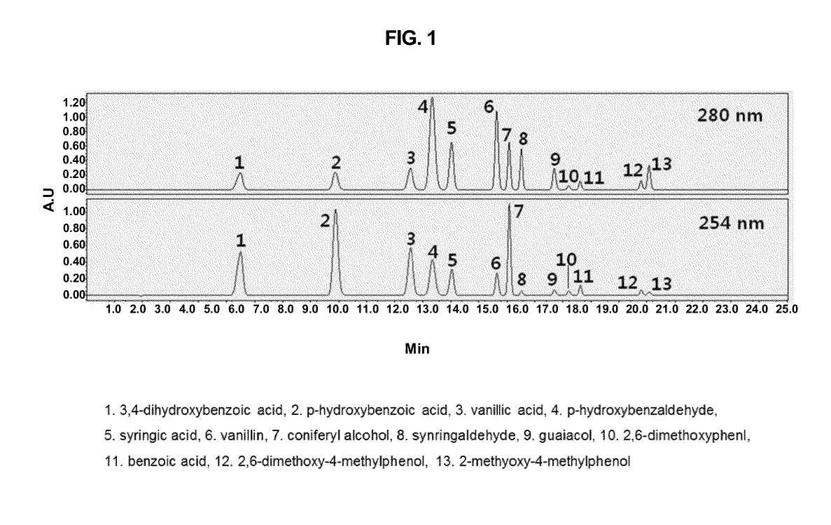

For use in the quantitative analysis of lignin-derived aromatic monomers, standard solutions of enzymatic reaction intermediates including p-hydroxybenzoic acid, benzoic acid and terephthalic acid were prepared, and analyzed using Waters e2695 HPLC equipped with a Waters 2489 UV/VIS (254 nm, 280 nm) detector (FIG. 1). For analysis, an)(Bridge C18 column (4.6×150 mm, 5 μm) was employed, and maintained at 35° C., with a mobile phase moved at 1 mL/min The mobile phase was a mixture of A) a 5% acetonitrile solution containing 0.1% formic acid, and B) a 50% acetonitrile solution containing 01% formic acid, and was applied in the following gradient elution manner: 1.5 min (0% B), 95 min (90% B), 16.5 min (40% B), 21.5 min (24% B), and 24.5 min (0% B). Prior to subsequent analysis, the column was pre-equilibrated for 6 min.

The production of terephthalic acid was monitored using HPLC and ESI-MS/MS (Waters TQD). This HPLC was conducted in the same condition as in the above HPLC. The condition for mass spectrometry was optimized with a standard solution of terephthalic acid. Mass spectra were obtained in the positive mode and the optimal condition for the spectrometry was set forth as follows: Capillary voltage: 3 kV, Cone voltage: 25 V, Source temperature: 120° C., Desolvation temperature: 300° C., Desolvation gas flow: 600 L/hr (N2), and Cone gas flow: 60 L/hr (N2). On the HPLC-ESI-MS/MS spectra, a peak for terephthalic acid was detected, in comparison with the standard solution, in the scan mode (50˜200, m/z) as molecular ions and specific fragment ions were generated at a given collision energy.

### Example 1

### Production of Benzoic Acid by Dehydroxylation

<Step 1> Construction of Recombinant Expression Vector Carrying Prephenate Hydro-Lyase (PHA2) Gene and Preparation of Transformed Microorganism

To produce prephenate hydro-lyase (PHA2), a PHA2 gene from S. cerevisiae was cloned. First, genomic DNA was isolated from S. cerevisiae ATCC 204508. On the basis of a nucleotide sequence (GenBank Accession Number; CAA86380.1) coding for a PHA2 gene, the following primers were designed:

Forward primer 1 (SEQ ID NO: 13):
5′-AAACATATG AAAATAAAAATTTTAGTAGA -3′
Reverse primer 1 (SEQ ID NO: 14):
5′-AAACTCGAG TTTGTGATAATATCTCTCAT -3′

The nucleotide sequence of PHA2 gene was amplified by PCR using the primers, with the genomic DNA of S. cerevisiae ATCC 204508 serving as a template.

A total volume of 50 μl of a PCR composition contained 100 ng of the template, 10 pmol of each primer, 2.5 mM dNTPs, a 1×PCR buffer, and a 2.5 U Taq polymerase. PCR started with pre-denaturation at 94° C. for 5 min, and was performed with 30 cycles of denaturation at 95° C. for 1 min; annealing at 54° C. for 30 sec; and elongation at 72° C. for 2 min, followed by post-polymerization at 72° C. for 5 min for final elongation.

The PCR product thus obtained was digested with NdeI/XhoI restriction enzymes, and inserted in the presence of T4 DNA ligase into the plasmid vector pET28a (Novagen) which was previously cut with the same enzymes, to construct a recombinant pET28a/PHA2 vector. PCR and cloning results were monitored by 1.2% agarose electrophoresis.

The recombinant expression vector was typically transformed into E. coli BL21 (DE3), and the transformant was cryo-preserved in 15% glycerol until use for enzyme expression.

<Step 2> Production of Prephenate Hydro-Lyase

To produce prephenate hydro-lyase in a large amount, the cryo-preserved recombinant E. coli was inoculated into 5 mL of LB broth in a test tube, and seed cultured at 37° C. with agitation to an absorbance of 2.5 at 600 nm. Then, the seed culture was added to 100 mL of LB broth in a 300 mL flask and cultured. When absorbance at 600 nm reached 0.6, 1 mM IPTG was added to induce the expression of the enzyme. In this regard, the cells were cultured at 30° C. with agitation at 250 rpm, and further incubated for 10 hrs after IPTG addition.

Then, the transformed cell culture was centrifuged at 4,000×g and 4° C. for 30 min, washed twice with a PBS buffer, mixed with a 50 mM Tris-HCl buffer (pH 7.5) before ultrasonic disruption. The cell lysate was again centrifuged at 13,000×g and 4° C. for 20 min, and the supernatant was withdrawn and subjected to Ni-NTA His-Tag chromatography to separate the enzyme. The bound enzyme was eluted with a 50 mM Tris-HCl buffer (pH 7.5) using a centrifugal separation filter (10 kDa). After concentration, the eluate was quantitatively analyzed using a protein assay (Bradford). Finally, the enzyme was obtained at a concentration of 5 mg/mL, and used in a enzymatic reaction with p-hydroxybenzoic acid as a substrate. Proteins were analyzed on 12% polyacrylamide gel by electrophoresis.

<Step 3> Production of Benzoic Acid

To 0.5 mL of a mixture containing 10 mM p-hydroxybenzoic acid (Sigma), 1 mM EDTA, 20 mM 2-mercaptoethanol, 50 μL of the 5 mg/L prephenate hydro-lyase purified in step 2 was added. After reaction for 5 hrs in a 37° C. incubator, three volumes of 1 N sodium hydroxide were added to the reaction mixture which was then filtered through a syringe filter (0.22 μm) to remove impurities. Concentrations of p-hydroxybenzoic acid and benzoic acid in the resulting sample were monitored using HPLC, and the results are given in Table 4.

TABLE 4

| | Before enzyme reaction | After enzyme reaction |
|---|---|---|
| | (mM) | (mM) |
| p-hydroxybenzoic acid | 9.98 | 2.46 |
| benzoic acid | 0.00 | 6.12 |

As shown in Table 4, p-hydroxybenzoic acid was converted into benzoic acid by prephenate hydro-lyase. The production of benzoic acid was confirmed by the mass spectroscopic analysis.

### Example 2

### Production of Terephthalic Acid by the Carboxylation Reaction

<Step 1> Construction of Recombinant Expression Vector Carrying 4-Hydroxybenzoic Acid Decarboxylase-Coding Gene and Preparation of Transformed Microorganism

To produce 4-hydroxybenzoic acid decarboxylase, genes of bsdB, bsdC and bsdD derived from B. substilis (strain 168) were cloned. It was reported that 4-Hydroxybenzoic acid decarboxylase catalyzed reversible reaction of the forward and reverse reaction depending on the reacting condition and the substrate (Can. J. Microbiol. 54: 75-81 (2008)). 4-Hydroxybenzoic acid decarboxylase was used for introducing the carboxyl group at 4-position of benzoic acid in this test. First, genomic DNA was isolated from B. substilis ATCC 6051. On the basis of a nucleotide sequence (GenBank Accession Number; bsdB:BAA08996.1., bsdC: BAA08997.1., bsdD: CAX52546.1) coding for the enzyme, the following primers were designed. 4-Hydroxybenzoic acid decarboxylase is a complex consisting of three kinds of domains of bsdB, bsdC and bsdD which are polycistronic gene located sequentially on the chromosome. Thus, the genes were cloned by using the following primers:

Forward primer 2 (SEQ ID NO: 15):
5′-AAACATATG AAAGCAGAATTCAAGCGTAA -3′
Reverse primer 2 (SEQ ID NO: 16):
5′-AAACTCGAG AGCCTTTCGTTCCGGCACCG -3′

The nucleotide sequence of the gene was amplified by PCR using the primers, with the genomic DNA of B. subtilis ATCC 6051 serving as a template.

A total volume of 50 μl of a PCR composition contained 100 ng of the template, 10 pmol of each primer, 2.5 mM dNTPs, a 1×PCR buffer, and a 2.5 U Taq polymerase. PCR started with pre-denaturation at 94° C. for 5 min, and was performed with 30 cycles of denaturation at 94° C. for 1 min; annealing at 55° C. for 30 sec; and elongation at 72° C. for 3 min, followed by post-polymerization at 72° C. for 5 min for final elongation.

The PCR product thus obtained was digested with NdeI/XhoI restriction enzymes, and inserted in the presence of T4 DNA ligase into the plasmid vector pET28a (Novagen) which was previously cut with the same enzymes, to construct a recombinant pET28a/PAD vector. PCR and cloning results were monitored by 1.5% agarose electrophoresis.

The recombinant expression vector was typically transformed into E. coli BL21 (DE3), and the transformant was cryo-preserved in 15% glycerol until use for enzyme expression.

<Step 2> Production of the Carboxylase

To produce the carboxylase in a large amount, the cryo-preserved recombinant E. coli was inoculated into 5 mL of LB broth in a test tube, and seed cultured at 37° C. with agitation to an absorbance of 2.5 at 600 nm. Then, the seed culture was added to 100 mL of LB broth in a 300 mL flask and cultured. When absorbance at 600 nm reached 0.6, 0.5 mM IPTG was added to induce the expression of the enzyme. In this regard, the cells were cultured at 33° C. with agitation at 250 rpm, and further incubated for 6 hrs after IPTG addition.

Then, the transformed cell culture was centrifuged at 4,000×g and 4° C. for 20 min, washed twice with a PBS buffer, mixed with a 50 mM Tris-HCl buffer (pH 7.5) before ultrasonic disruption. The cell lysate was again centrifuged at 13,000×g and 4° C. for 20 min, and the supernatant was withdrawn and subjected to Ni-NTA His-Tag chromatography to purify the enzyme. The bound enzyme was eluted with a 50 mM Tris-HCl buffer (pH 7.5) using a centrifugal separation filter (10 kDa). After concentration, the eluate was quantitatively analyzed using a protein assay (Bradford). Finally, the enzyme was obtained at a concentration of 5 mg/mL, and used in enzymatic reaction.

<Step 3> Production of Terephthalic Acid

To 0.5 mL of a mixture containing 10 mM benzoic acid (Sigma), 100 mM sodium bicarbonate, 50 μL of the 5 mg/L the Carboxylase purified in step 2 was added and then reacted for 5 hours in the 37° C. anaerobic chamber. After reaction termination, three volumes of aqueous solution of 1 N sodium hydroxide were added to the reaction mixture which was then filtered through a syringe filter (0.22 μm) to remove impurities. Concentrations of reduced benzoic acid and produced terephthalic acid in the resulting sample were monitored using HPLC, and the results are given in Table 5.

TABLE 5

| | Before enzyme reaction | After enzyme reaction |
|---|---|---|
| | (mM) | (mM) |
| benzoic acid | 10.32 | 4.81 |
| Terephthalic acid | 0.00 | 3.24 |

As shown in Table 5, benzoic acid was converted into terephthalic acid by 4-Hydroxybenzoic acid decarboxylase. The production of terephthalic acid was confirmed by the mass spectroscopic analysis.

### Example 3

### Production of Terephthalic Acid from p-Hydroxybenzoic Acid

The one-pot enzyme reaction using the Dehydroxylase in Example 1 and the Carboxylase in Example 2 are used for terephthalic acid from p-hydroxybenzoic acid.

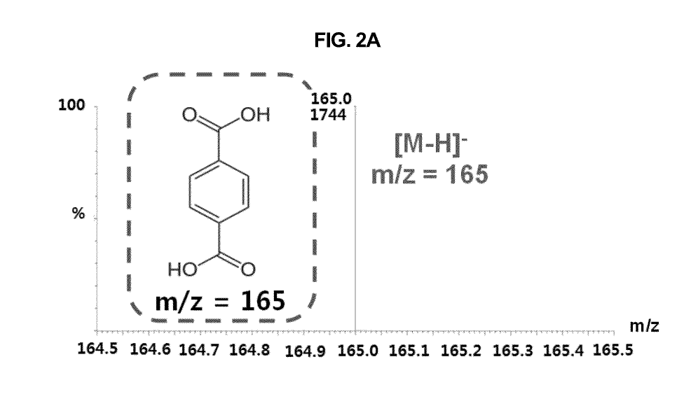

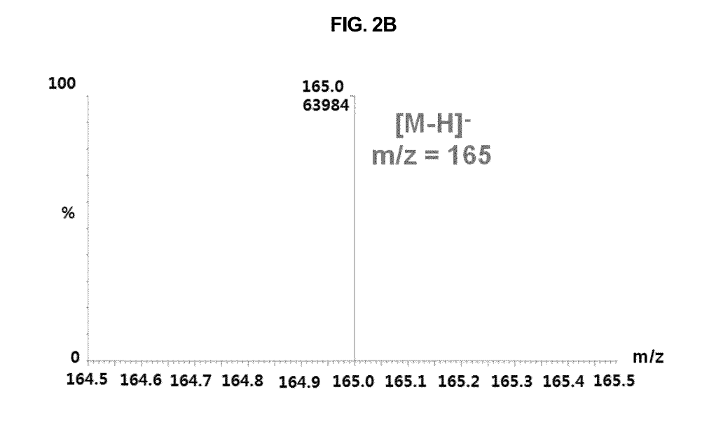

50 μL (5 mg/mL) each of the dehydroxylase in Example 1 and the Carboxylase in Example 2 was to 0.5 mL of a mixture containing 400 μL of 20 mM p-hydroxybenzoic acid (Sigma) dissolved in 100 mM of sodium bicarbonase (pH 8.5), and then reacted for 5 hrs in a 37° C. incubator. After the reaction termination, three volumes of 1 N sodium hydroxide were added to the reaction mixture which was then filtered through a syringe filter (0.22 μm) to remove impurities. Concentrations of reduced p-hydroxybenzoic acid and produced terephthalic acid in the resulting sample were monitored using HPLC, and the results are given in Table 6. The produced terephthalic acid was confirmed by mass spectrometry (FIGS. 2A-2B).

TABLE 6

| | Before enzyme reaction | After enzyme reaction |
|---|---|---|
| | (mM) | (mM) |
| p-hydroxybenzoic acid | 19.59 | 2.93 |
| benzoic acid | 0.00 | 3.48 |
| Terephthalic acid | 0.00 | 7.29 |

### Example 4

### Production of Aminodeoxychorismate Lyase (ADC Lyase)

<Step 1> Construction of Recombinant Expression Vector Carrying Aminodeoxychorismate Lyase Gene and Preparation of Transformed Microorganism

To produce aminodeoxychorismate lyase (ADC lyase), an ADC lyase gene from S. cerevisiae was cloned. First, genomic DNA was isolated from S. cerevisiae KCCM 50712. On the basis of a nucleotide sequence (GenBank Accession Number; DAA10190.1) coding for the ADC lyase of S. cerevisiae KCCM 50712, the following primers were designed:

Forward primer 3 (SEQ ID NO: 17):
5′-AAACATATG TCACTAATGGACAATTGGAA-3
Reverse primer 3 (SEQ ID NO: 18):
5′- AAACTCGAG ATATTTTGTCTTCACTGTTC-3′

The nucleotide sequence of ADC lyase gene was amplified by PCR using the primers, with the genomic DNA of S. cerevisiae KCCM 50712 serving as a template.

A total volume of 50 μl of a PCR composition contained 100 ng of the template, 10 pmol of each primer, 2.5 mM dNTPs, a 1×PCR buffer, and a 2.5 U Taq polymerase. PCR started with pre-denaturation at 94° C. for 5 min, and was performed with 30 cycles of denaturation at 94° C. for 1 min; annealing at 55° C. for 30 sec; and elongation at 72° C. for 3 min, followed by post-polymerization at 72° C. for 5 min for final elongation.

The PCR product thus obtained was digested with NdeI/XhoI restriction enzymes, and inserted in the presence of T4 DNA ligase into the plasmid vector pET28a (Novagen) which was previously cut with the same enzymes, to construct a recombinant pET28a/ADCL vector. PCR and cloning results were monitored by 1.2% agarose electrophoresis.

The recombinant expression vector was typically transformed into E. coli BL21 (DE3), and the transformant was cryo-preserved in 15% glycerol until use for enzyme expression.

<Step 2> Production of ADC Lyase

To produce ADC lyase in a large amount, the cryo-preserved recombinant E. coli was inoculated into 5 mL of LB broth in a test tube, and seed cultured at 37° C. with agitation to an absorbance of 2.5 at 600 nm. Then, the seed culture was added to 100 mL of LB broth in a 300 mL flask and cultured. When absorbance at 600 nm reached 0.6, 0.5 mM IPTG was added to induce the expression of the enzyme. In this regard, the cells were cultured at 33° C. with agitation at 250 rpm, and further incubated for 6 hrs after IPTG addition.

Then, the transformed cell culture was centrifuged at 4,000×g and 4° C. for 20 min, washed twice with a PBS buffer, mixed with a 50 mM Tris-HCl buffer (pH 7.5) before ultrasonic disruption. The cell lysate was again centrifuged at 13,000×g and 4° C. for 20 min, and the supernatant was withdrawn and subjected to Ni-NTA His-Tag chromatography to purify the enzyme. The bound enzyme was eluted with a 50 mM Tris-HCl buffer (pH 7.5) using a centrifugal separation filter (10 kDa). After concentration, the eluate was quantitatively analyzed using a protein assay (Bradford). Finally, the enzyme was obtained at a concentration of 5 mg/mL, and used in enzymatic reaction.

### Example 5

### Production of Terephthalic acid by Enzymatic Reaction Using Lignin Degradation Product as Substrate

Lignin was degraded using a laboratory high-pressure reactor (450 mL, Parr 4562). A reactant with a lignin content of 5.0% (w/v) was prepared by adding 10.0 g of graft lignin to 200 mL of 1 M NaOH. The reactant was further mixed with 10 g of the catalyst KMnO₄, loaded to a stainless steel high-pressure reactor with an internal volume of 450 mL, sealed, and stirred at a speed of 500 rpm. After the reactor was filled with oxygen gas at a pressure of about 5 bar for 2 min via a sampling line communicating with the interior thereof; it was heated. When the internal temperature of the reactor reached 140° C., the reaction was continued for 60 min. The reaction temperature was adjusted by a PID controller through a cooling water tube. At 60 min of the reaction, a sample was withdrawn via a sampling line, and then, the reaction was terminated.

2 mL of the sample containing alkaline breakdown products of lignin was 3-fold diluted in 4 mL of distilled water, followed by removal of lignin by filtration (10 kDa MWCO). To 1 mL of the lignin-free sample were added 9 volumes of methanol, and lignin breakdown products were purified by filtration through a syringe filter (0.22 μm).

The lignin breakdown products obtained above were enzymatically converted into terephthalic acid. In this regard, a solution of the lignin breakdown products was adjusted to pH 8.0 using a small amount of 10 M HCl, and filtered through a 10 kDa MWCO filter. The filtrate was used as a substrate in reaction with a mixture of 50 μL of 5 mg/mL aminodeoxychorismate lyase (ADC lyase), obtained in Example 4, and 50 μL of 5 mg/ml aldehyde dehydrogenase (Sigma A6338, ALD4) for 3 hrs in a 37° C. incubator. Of the reaction mixture, 50 μL was withdrawn and used for analysis while the remainder was used as a substrate for a subsequent enzyme reaction. For p-hydroxybenzoic acid analysis, the sample was extracted with 9 volumes of methanol (450 μL), and filtered through a syringe filter (0.22 μm). The resulting sample was analyzed for p-hydroxybenzoic acid by HPLC (Table 7).

The Dehydroxylase of Example 1 and the Carboxylase of Example 2 was added at a final concentration of 500 μg/mL respectively, to 400 μL of the remainder enzyme reaction mixture which was then mixed with sodium bicarbonate at a final concentration of 100 mM, followed by reaction for 10 hrs in a 37° C. anaerobic chamber. After completion of the reaction, the sample was filtered through a syringe filter (0.22 μm) to remove impurities. Analysis results of the sample are summarized in Table 7.

TABLE 7

| | | Product obtained after | |
|---|---|---|---|
| | | treatment with | Product obtained after |
| | Lignin | aldehyde | treatment with |
| | degradation | dehydrogenase and | dehydroxylase and |
| Compound | Product (mM) | ADC lyase (mM) | Carboxylase (mM) |
| p-Hydroxybenzoic acid | 0.21 | 4.53 | 1.35 |
| p-Hydroxybenzaldehyde | 0.91 | 0.18 | 0.00 |
| Vanillic acid | 2.40 | 1.95 | 1.24 |
| Vanillin | 5.82 | 2.98 | 1.32 |
| Syringic acid | 0.50 | 0.12 | 0.00 |
| Syrinaldehyde | 0.21 | 0.00 | 0.00 |
| Benzoic acid | 0.00 | 0.00 | 0.52 |
| Terephthalic acid | 0.00 | 0.00 | 1.16 |

Although the preferred embodiments of the present invention have been disclosed for illustrative purposes, those skilled in the art will appreciate that various modifications, additions and substitutions are possible, without departing from the scope and spirit of the invention.

What is claimed is:

1. A method of biologically producing an terephthalic acid or a derivative thereof represented by Chemical Formula 1, comprising contacting a substrate containing an aromatic carboxylic acid represented by Chemical Formula 2 with a biocatalyst that adds a carboxyl group at para-position of the aromatic carboxylic acid: 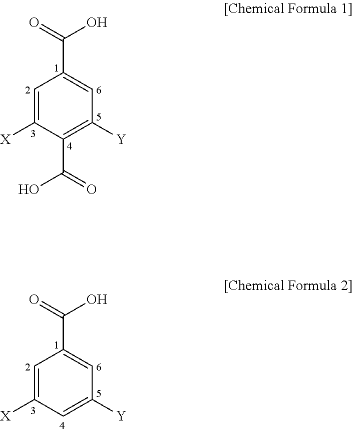
wherein, X and Y are independently hydrogen, hydroxy, or C₁-C₄ alkoxy.

2. The method of claim 1, wherein X and Y are not simultaneously hydrogen in Chemical Formula 2, and the method further comprises contacting the substrate with a biocatalyst that removes a substituent selected from the group consisting of a hydroxyl group and a C₁-C₄ alkoxy group located at position 3, position 5, or both of positions 3 and 5 of the benzene ring, before, simultaneously with, or after contacting with the biocatalyst that adds a carboxyl group at para-position of the aromatic carboxylic acid.

3. The method of claim 1, wherein the substrate also comprises an aromatic carboxylic acid of Chemical Formula 3 having a para-hydroxy group, and the method further comprises contacting the substrate with a biocatalyst that removes the para-hydroxy group from the aromatic carboxylic acid of Chemical Formula 3, before, simultaneously with, or after contacting with the biocatalyst that adds a carboxyl group at para-position of the aromatic carboxylic acid of Chemical Formula 2: 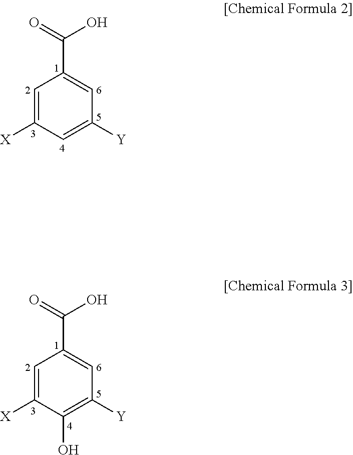
wherein, X and Y are independently hydrogen, hydroxy, or C₁-C₄ alkoxy.

4. The method of claim 3, wherein X and Y are not both hydrogen in Chemical Formula 3, and the method further comprises contacting the substrate with a biocatalyst that removes a substituent selected from the group consisting of hydroxy and C₁-C₄ alkoxy located at position 3, position 5, or both of positions 3 and 5 of the benzene ring, before, simultaneously with, or after contacting with the biocatalyst that removes the hydroxy group at para-position.

5. The method of claim 1, wherein the biocatalyst is an enzyme, a microorganism that produces the enzyme, a lysate of the microorganism, or an extract from the lysate of the microorganism.

6. The method of claim 5, wherein the biocatalyst is a wild-type microorganism or a recombinant microorganism.

7. The method of claim 1, wherein contacting the substrate with the biocatalyst is comprises contacting the substrate with an enzyme, a microorganism containing the enzyme, a lysate of the microorganism, or an extract from the lysate of the microorganism, or by culturing the microorganism in a medium containing the substrate.

8. The method of claim 1, wherein the biocatalyst that adds a carboxyl group at the para-position of the aromatic carboxylic acid comprises at least one enzyme selected from the group consisting of aminobenzoate decarboxylase (EC 4.1.1.24), 3-Hydroxy-2-methylpyridine-4,5-dicarboxylate 4-decarboxylase (EC 4.1.1.51), 4,5-Dihydroxyphthalate decarboxylase (EC 4.1.1.55), Orsellinate decarboxylase (EC 4.1.1.58, Gallate decarboxylase (EC 4.1.1.59), 4-hydroxybenzoate decarboxylase (EC 4.1.1.61), Protocatechuate decarboxylase (EC 4.1.1.63), 3,4-dihydroxyphthalate decarboxylase (EC 4.1.1.69) phenylphosphate carboxylase (EC 4.1.1.x); a microorganism that produces at least one of the foregoing enzymes; a lysate of a microorganism containing at least one of the foregoing enzymes; or an extract from the lysate of the microorganism containing at least one of the foregoing enzymes.

9. The method of claim 3, wherein the biocatalyst that removes para-hydroxy comprises at least one enzyme selected from the group consisting of bile-acid 7-alpha-dehydroxylase (EC 1.17.99.5), 4-hydroxybenzoyl-CoA reductase (EC 1.3.7.9), 3-dehydroquinate hydro-lyase (EC 4.2.1.10), aldos-2-ulose dehydratase (EC 4.2.1.110), o-succinylbenzoate synthase (EC 4.2.1.113), 3-dehydroshikimate hydro-lyase (EC 4.2.1.118), prephenate hydro-lyase (EC 4.2.1.51), arogenate dehydratase (EC 4.2.1.91), scytalone 7,8-hydro-lyase (EC 4.2.1.94), 16a-hydroxyprogesterone hydro-lyase (EC 4.2.1.98); a microorganism that produces at least one of the foregoing enzymes; a lysate of a microorganism containing at least one of the foregoing enzymes; or an extract from the lysate of the microorganism containing at least one of the foregoing enzymes.

10. The method of claim 2, wherein the biocatalyst that removes at least one substituent selected from the group consisting of hydroxy and C₁-C₄ alkoxy is at least one enzyme selected from the group consisting of anthranilate synthase (EC 4.1.3.27), aminodeoxychorismate lyase (EC 4.1.3.38), chorismate lyase (EC 4.1.3.40), 3-dehydroquinate hydro-lyase (EC 4.2.1.10), 3-dehydroshikimate hydro-lyase (EC 4.2.1.118), prephenate hydro-lyase (EC 4.2.1.51), 5-041-carboxyvinyl)-3-phosphoshikimate phosphate-lyase (EC 4.2.3.5), isochorismate lyase (EC 4.2.99.21), hydroxyphenylpyruvate synthase (EC 5.4.99.5); a microorganism that produces at least one of the foregoing enzymes; a lysate of a microorganism containing at least one of the foregoing enzymes; or an extract from the lysate of the microorganism containing at least one of the foregoing enzymes.

11. The method of claim 4 wherein the biocatalyst that removes at least one substituent selected from the group consisting of hydroxy and C₁-C₄ alkoxy is at least one enzyme selected from the group consisting of anthranilate synthase (EC 4.1.3.27), aminodeoxychorismate lyase (EC 4.1.3.38), chorismate lyase (EC 4.1.3.40), 3-dehydroquinate hydro-lyase (EC 4.2.1.10), 3-dehydroshikimate hydro-lyase (EC 4.2.1.118), prephenate hydro-lyase (EC 4.2.1.51), 5-041-carboxyvinyl)-3-phosphoshikimate phosphate-lyase (EC 4.2.3.5), isochorismate lyase (EC 4.2.99.21), hydroxyphenylpyruvate synthase (EC 5.4.99.5); a microorganism that produces at least one of the foregoing enzymes; a lysate of a microorganism containing at least one of the foregoing enzymes; or an extract from the lysate of the microorganism containing at least one of the foregoing enzymes.

12. The method of claim 1, wherein the substrate comprises an aromatic aldehyde of Chemical Formula 4, and the method further comprises
converting the aromatic aldehyde of Chemical Formula 4 to an aromatic carboxylic acid of Chemical Formula 3 by chemical oxidation or biocatalytic oxidation;
and contacting the aromatic carboxylic acid of Chemical Formula 3 with a biocatalyst that removes the para-hydroxy group from the aromatic carboxylic acid of Chemical Formula 3 to provide the aromatic carboxylic acid of Chemical Formula 2: 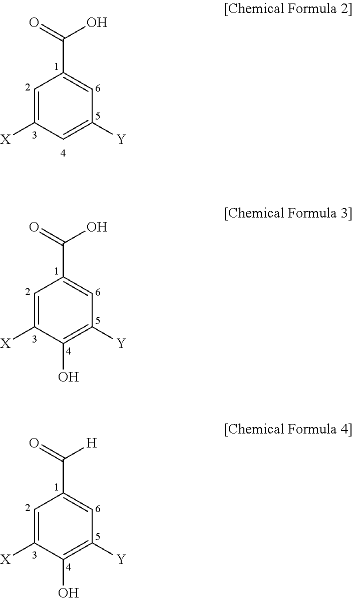
wherein, X and Y are independently hydrogen, hydroxy, or C₁-C₄ alkoxy.

13. The method of claim 12, wherein X and Y are not simultaneously hydrogen in Chemical Formula 4, and the method further comprises contacting the substrate with a biocatalyst that removes a substituent selected from the group consisting of hydroxy and C₁-C₄ alkoxy located at position 3, position 5, or both of positions 3 and 5 of the benzene ring before, simultaneously with, or after the oxidation of the aromatic aldehyde of Chemical Formula 4.

14. The method of claim 3, the compounds represented by Chemical Formula 3 and Chemical Formula 4 are derived from lignin.

15. The method of claim 12, the compounds represented by Chemical Formula 3 and Chemical Formula 4 are derived from lignin.

16. The method of claim 15, wherein the substrate containing an aromatic carboxylic acid represented by Chemical Formula 2, is obtained by:
degrading lignin to give a lignin degradation product comprising an aromatic carboxylic acid having a para-hydroxy group of Chemical Formula 3; and
contacting the lignin degradation product with the biocatalyst that removes a para-hydroxy group from the benzene ring of Chemical Formula 3 to provide an aromatic carboxylic acid represented by Chemical Formula 2.

17. The method of claim 16, wherein X and Y are not simultaneously hydrogen in Chemical Formula 3, and the method further comprises contacting the substrate with a biocatalyst that removes a substituent selected from the group consisting of hydroxy and C₁-C₄ alkoxy located at positions 3, position 5, or both of positions 3 and 5 of the benzene ring, before, simultaneously with, or after contacting with the biocatalyst that removes the para-hydroxy group.

18. The method of claim 16, further comprising converting an aromatic aldehyde represented by Chemical Formula 4 to the aromatic carboxylic acid of Chemical Formula 3 by chemical oxidation or biocatalytic oxidation, before or simultaneously with contacting with the biocatalyst that removes the para-hydroxy group.

19. The method of claim 16, wherein the degradation of lignin is carried out using at least one selected from the group consisting of pyrolysis, gasification, hydrogenolysis, acidolysis, alkaline lysis, chemical oxidation, hydrolysis under supercritical conditions, and enzymolysis.

20. A polyester produced from the terephthalic acid or derivatives thereof obtained in claim 1.

21. A method of biologically producing a terephthalic acid or a derivative thereof represented by Chemical Formula 1, the method comprising
contacting a substrate containing an aromatic carboxylic acid of Chemical Formula 3 having a para-hydroxy group with a biocatalyst that removes the para-hydroxy group to provide an aromatic carboxylic acid of Chemical Formula 2;
and contacting the aromatic carboxylic acid of Chemical Formula 2 with a biocatalyst that adds a carboxyl group at para-position of the aromatic carboxylic acid to provide a terephthalic acid or a derivative thereof represented by Chemical Formula 1: 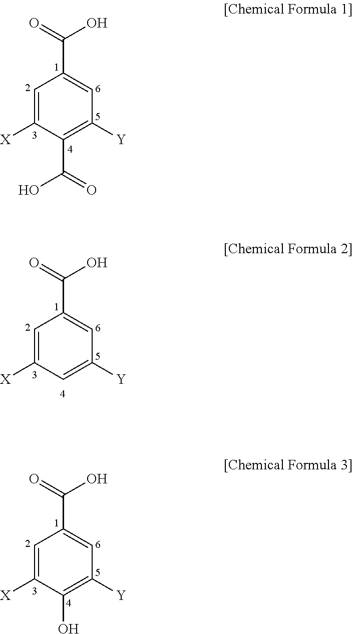
wherein X and Y are independently hydrogen, hydroxy, or C₁-C₄ alkoxy.

22. A method of biologically producing a terephthalic acid or a derivative thereof represented by Chemical Formula 1, the method comprising
providing a substrate comprising an aromatic aldehyde of Chemical Formula 4;
converting the aromatic aldehyde of Chemical Formula 4 to an aromatic carboxylic acid of Chemical Formula 3 by chemical oxidation or biocatalytic oxidation;
contacting the aromatic carboxylic acid of Chemical Formula 3 having a para-hydroxy group with a biocatalyst that removes the para-hydroxy group to provide an aromatic carboxylic acid of Chemical Formula 2;
and contacting the aromatic carboxylic acid of Chemical Formula 2 with a biocatalyst that adds a carboxyl group at para-position of the aromatic carboxylic acid to provide a terephthalic acid or a derivative thereof represented by Chemical Formula 1: 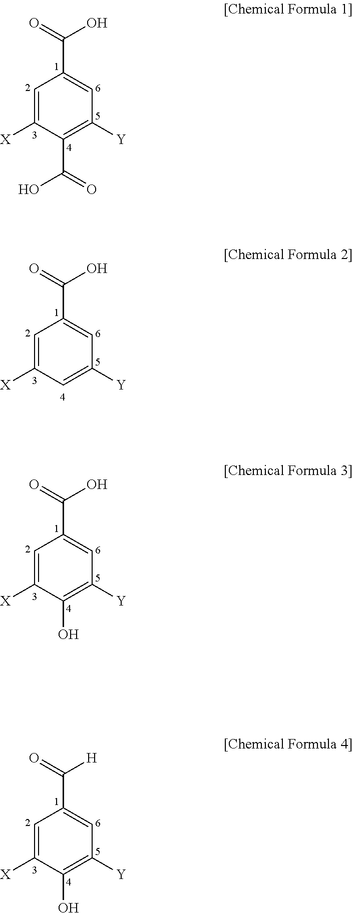
wherein X and Y are independently hydrogen, hydroxy, or C₁-C₄ alkoxy.
